(19) United States
(12) Patent Application Publication
(10) Pub. No.: US 2008/0067462 A1
(43) Pub. Date: Mar. 20, 2008

(54) Stopcock With Swabbable Valve

(76) Inventors: Pavel T. Miller, St. Petersburg, FL (US); Stephen J. Rogier, Palm Harbor, FL (US)

Correspondence Address: HOLLAND & KNIGHT LLP;ATTN: STEFAN V. STEIN/ IP DEPT., POST OFFICE BOX 1288, TAMPA, FL, 33601-1288, US

(21) Appl. No.: 11/836,689

(22) Filed: Aug. 9, 2007

Related U.S. Application Data
(60) Provisional application No. 60/836,598, filed on Aug. 9, 2006.

(51) Int. Cl. A61M 39/22

(52) U.S. Cl. 251/149.1

(57) ABSTRACT

A stopcock for connection in-line with a fluid line including a main valve body main having at least a plurality of inlets, including a stopcock valve element positioned within the main valve body that controls the flow of fluid through the inlets and a swabbable valve fitted to one of the inlets.

### CROSS-REFERENCE TO RELATED INVENTIONS

This application claims the benefit of provisional application number 60/836,598, filed Aug. 9, 2006, the disclosure of which is hereby incorporated by reference herein.

### BACKGROUND OF THE INVENTION

1. Field of the Invention

This invention relates to valves. More particularly, this invention relates to swabbable valves used in the medical industry.

2. Description of the Background Art

As the term implies, the term “swabbable” is often used in conjunction with medical valves having a female luer fitting configured in such a way that allows swabbing of the exposed portion of the valve just prior to connection with a male luer needleless syringe or other device.

Representative swabbable valves are disclosed in U.S. Pat. Nos. 6,036,171, 6,692,478, 6,221,065, 6,168,137, 6,117,114, 6,651,956, 6,113,068 and RE37,357, the disclosures of each of which are hereby incorporated by reference herein.

As taught by the above-referenced patents, swabbable valves are often employed in IV-sets for needleless interconnection of an IV bag and its associated tubing to deliver medicine to a patient intravenously. Such needleless interconnection occurs with each device being connected end to end.

Swabbable valves preferably satisfy main requirements. For example, they should safely withstand, without loss of performance, at least 100 connects and disconnects to an injection site before the set is replaced. In addition, a connection is maintained for an extended period of time before disconnection is made. The site should still be capable of accepting subsequent connections without allowing any leakage. Valves should seal against pressurized fluid within a set. They should withstand pressures in excess of, for example, 30 PSI for a short time, such as during an injection made through an adjacent site or if a pump is connected in the circuit. Further, valves should be capable of being manufactured at high speeds and low costs. At the same time, the design must allow for minimal manufacturing defects. Still further, it is desirable that such valves have as few components as possible, and be easily assembled, without requiring any difficult component orientation or positioning.

With particular regard to medical applications, valves should not contain any dead space where fluid can collect and not be readily flushed away. Also, priming volume should be minimized. Furthermore, valves should also be easily accessible by standard luer connectors and provide secure locking features, so they could be left connected to the site without further assistance from a practitioner.

Another highly-desirable feature is easy and safe swabbability of the valve inlet area. Unfortunately, most current swabbable valves restrict free flow of passing fluid by employing narrow passages, ribs or internal cannula-like features. Restricting the flow path in such a manner may create conditions for hemolytic damage. Such restrictions also make the valve generally more difficult to flush.

Indeed, in valves used for blood sampling, there is a need for valves that do not have any space where fluid could collect and stagnate. The valve should be fully flushable to preclude thrombosis that might otherwise occur in dead spaces even of minimal sizes.

Furthermore, in medical applications, it is usually desirable to prevent the patient from being exposed to the fluid which is being injected to or extracted from the patient, and it is desirable to insulate nurses and doctors from exposure to the liquid which may contain the patient's blood or waste products. However, often the instrument used to inject or withdraw the fluid (which is generally the male component of the syringe), retains some of the fluid on the tip thereof, thus providing a risk to nurses and doctors of being exposed to the fluid. Wiping off this fluid prior to disconnecting the instrument is highly desirable.

As taught for example in U.S. Pat. Nos. 6,221,065 and 6,117,114, the disclosures of each of which are hereby incorporated by reference herein, Y-site connectors are commonly used in IV-sets. U.S. Pat. No. RE37,357 describes a valve in the form of a T-port where resulting flow from the swabbable end is very limited. Such resistance to the flow creates an undesirable condition for leaks around the access instrument's tip. A swabbable valve used as an injection port or a sampling port should pose minimum resistance to the flow from the syringe or communicating line. Restrictive valve geometry means slow fluid delivery and if there is blood in the fluid, there is the possibility of hemolytic damage caused by high flow speeds at narrow or curved passages.

There presently exists a need for a swabbable valve incorporated into a stopcock providing in-line access to IV tubing.

Therefore, it is an object of this invention to provide an improvement which overcomes the aforementioned inadequacies of the prior art devices and provides an improvement which is a significant contribution to the advancement of the stopcock art.

Another object of this invention is to provide a swabbable valve allowing needleless in-line access to IV tubing.

Another object of this invention is to provide a swabbable valve incorporated into a stopcock facilitating needleless in-line access to medical tubing.

Another object of this invention is to provide stopcock for connection in-line with a fluid line, comprising a main valve body main having at least a plurality of inlets, a stopcock valve element positioned within the main valve body that controls the flow of fluid through the inlets and a swabbable valve fitted to one of the inlets.

The foregoing has outlined some of the pertinent objects of the invention. These objects should be construed to be merely illustrative of some of the more prominent features and applications of the intended invention. Many other beneficial results can be attained by applying the disclosed invention in a different manner or modifying the invention within the scope of the disclosure. Accordingly, other objects and a fuller understanding of the invention may be had by referring to the summary of the invention and the detailed description of the preferred embodiment in addition to the scope of the invention defined by the claims taken in conjunction with the accompanying drawings.

### BRIEF DESCRIPTION OF THE DRAWINGS

For a fuller understanding of the nature and objects of the invention, reference should be had to the following detailed description taken in connection with the accompanying drawings in which:

FIG. 1 is a top view of the swabbable stopcock of the invention;

FIG. 2 is a side view of FIG. 1;

FIG. 3 is a cross-sectional view of FIG. 2 along lines 3-3;

FIG. 4 is a top perspective view of the main valve body;

FIG. 5 is a cross-sectional view of FIG. 4 along lines 5-5;

FIG. 6 is a top perspective view of the stopcock valve element;

FIG. 7 is a bottom perspective view of the stopcock valve element;

FIG. 8 is a top view of the stopcock valve element;

FIG. 9 is a bottom view of the stopcock valve element;

FIG. 10 is a cross-sectional view of FIG. 8 along lines 10-10;

FIG. 11 is a cross-sectional view of FIG. 8 along lines 11-11;

FIG. 12 is a longitudinal cross-sectional view of the collar of the male luer fitting;

FIG. 13 is a perspective view of swabbable valve element;

FIG. 14 is a longitudinal cross-sectional view of the swabbable valve element; and

FIG. 16 is a longitudinal cross-sectional view of the female collar of the swabbable valve.

Similar reference characters refer to similar parts throughout the several views of the drawings.

### DETAILED DESCRIPTION OF THE PREFERRED EMBODIMENT

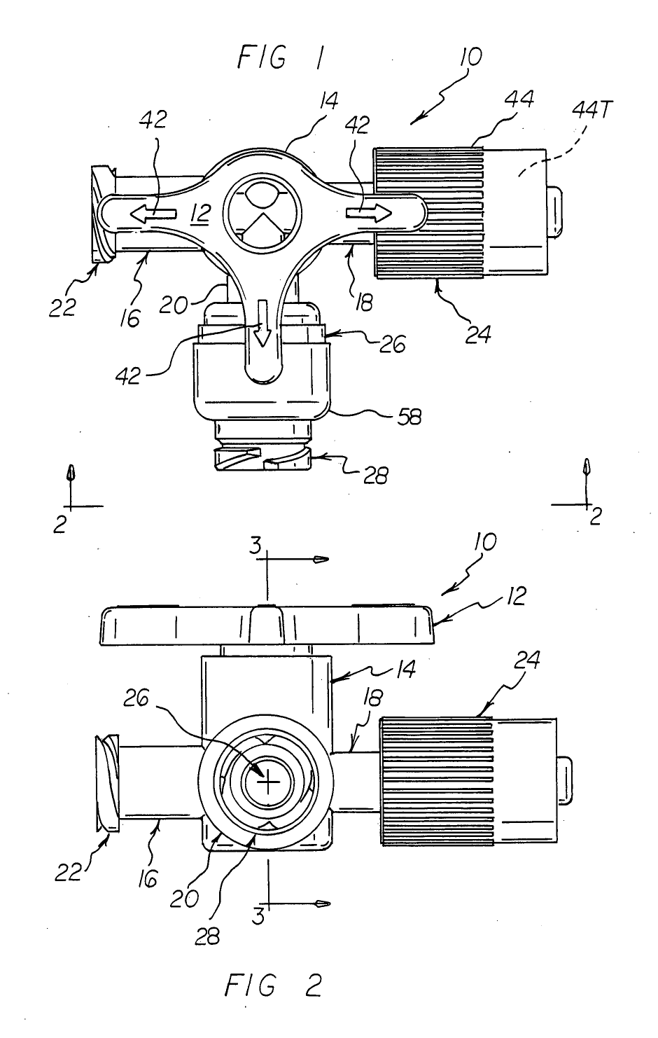

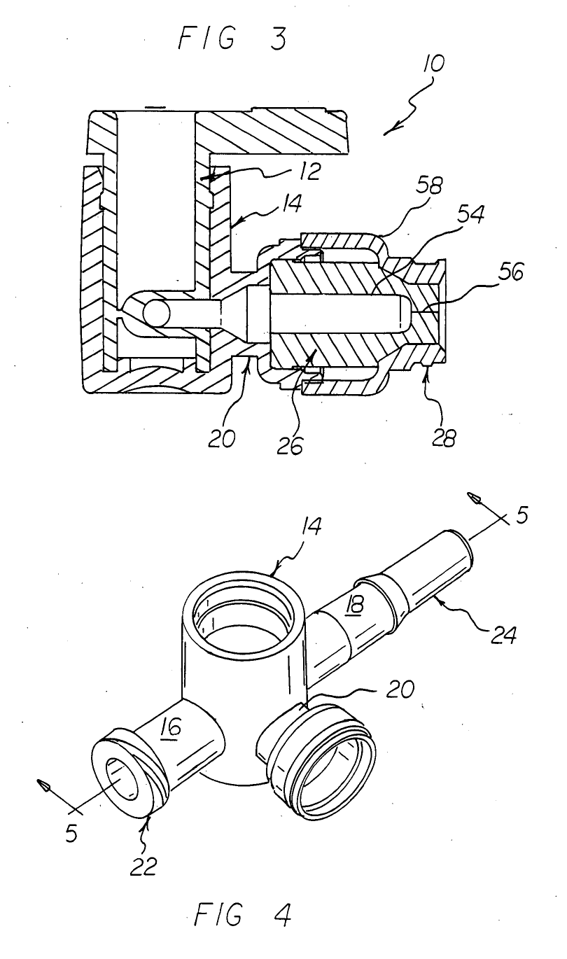

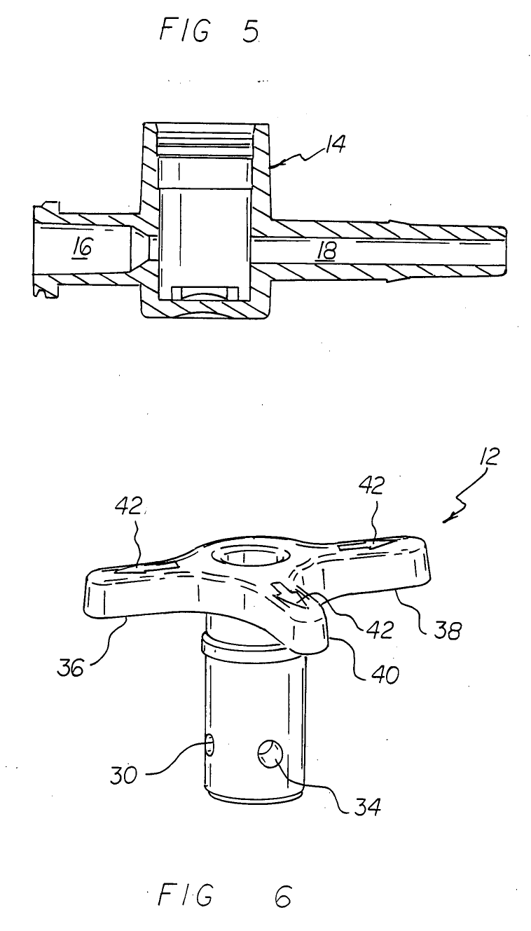

As shown in the FIGS. 1-3, the swabbable stopcock 10 of the invention comprises a stopcock valve element 12 that controls the flow of fluid through a main valve body 14 having at least two inlets, preferably three, 16, 18 and 20 (see also FIGS. 4 and 5). One of the inlets 16, 18 or 20 (e.g., inlet 16) is fitted with a conventional female luer fitting 22. Another one of the inlets 16, 18 and 20 (e.g., inlet 18) is fitted with a male luer fitting 24. Still another one of the inlets 16, 18 and 20 (e.g., inlet 20) is fitted with a swabbable valve 26 having a luer fitting 28. Preferably, the female/male luer inlets 16 and 18 are positioned in-line and the inlet 20 comprising the swabbable valve 26 is positioned transversely therebetween. The stopcock 10 may be connected in-line with an IV line via the respective female & male luer fittings 22 and 24 of the in-line inlets 16 and 18. With the swabbable valve 26 of the middle inlet 20 comprising a normally-closed valve, the arrangement allows the stopcock 10 to be used in-line in the IV set to maintain a “closed” system to prevent any leakage from the IV line.

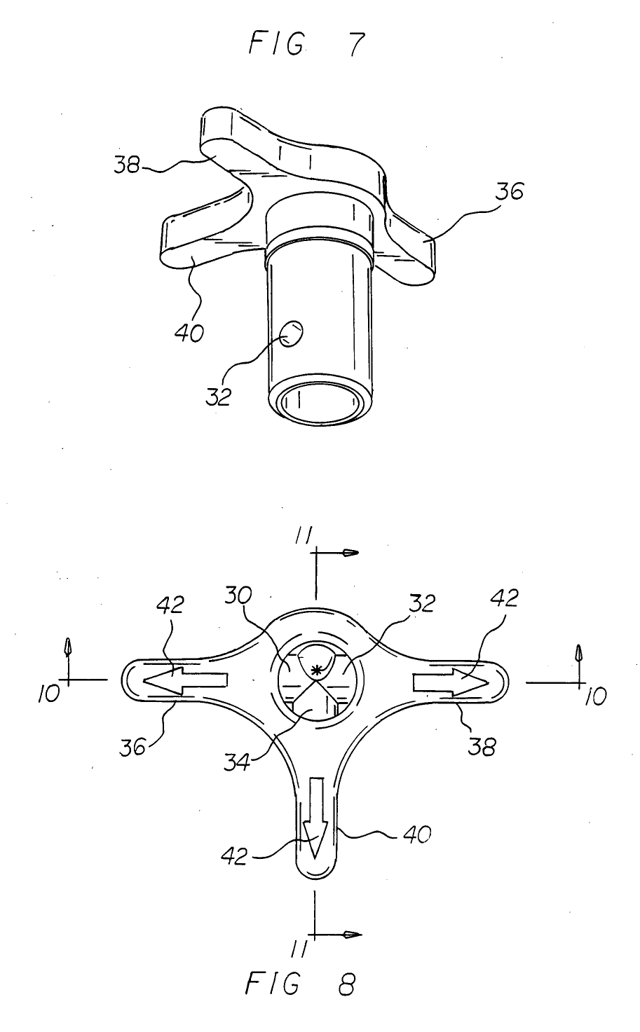

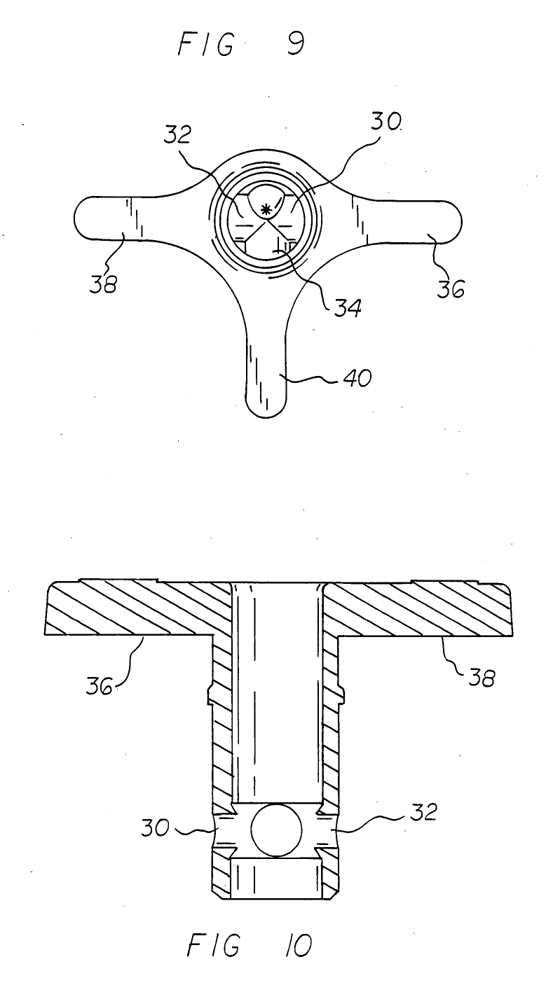

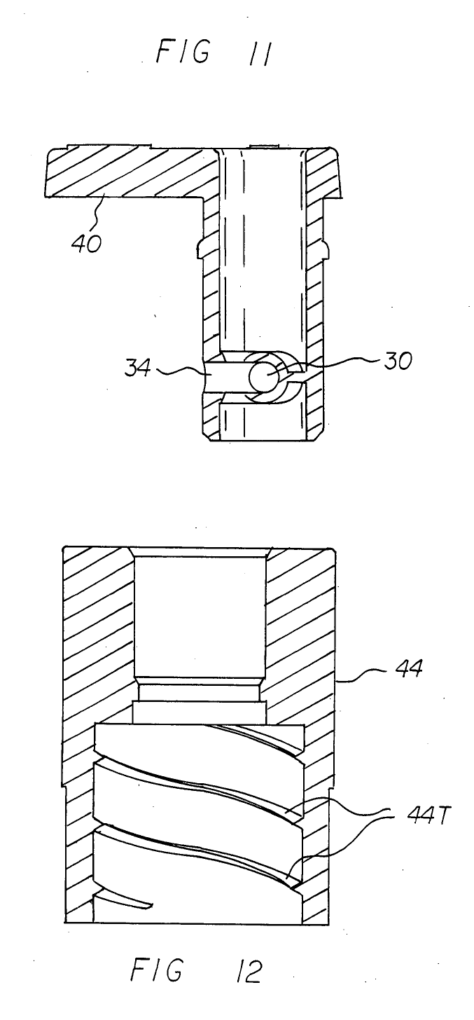

As best shown in FIGS. 6-11, the stopcock valve element 12 comprises an elongated circular cylindrical configuration that is dimensioned to sealingly snap-fit into the lumen of the central circular cylindrical bore of the main body member 14. The stopcock valve element 12 comprises three flow channels 30, 32 and 34(described hereinafter) formed in the lower portion thereof in a position such that when the stopcock valve element 12 is positioned fully into the main body element 12, the plane of the three flow channels 30, 32 and 34 are alignment with the plane of the three inlets 16, 18 and 20 of the valve body 14.

The stopcock valve element 12 further comprises three handles 36, 38 and 40 extending from the upper portion of the elongated circular cylindrical configuration thereof in radial alignment with the respective flow channels 30, 32 and 34 (i.e., handle 36 is radially aligned with channel 30, handle 38 is radially aligned with channel 32 and handle 340 is radially aligned with channel 334).

As shown in FIG. 12, the male luer 24 of inlet 18 comprises a conventional locking collar 44 that snap-fits onto the inlet 18 in a conventional manner. The locking collar 44 comprises internal threads 44T that allows threaded engagement with a female luer fitting.

The swabbable valve 26 of inlet 20 functions to preclude any fluid flow through inlet 20 until it is actuated open by a device (e.g., the plain tip of a syringe or the luer tip of a syringe, IV fitting, IV bag, etc.). The swabbable valve 26 may comprise any type of a swabbable valve such as those disclosed above. Preferably, however, swabbable valve 20 comprises a split septum type of valve manufactured by Halkey-Roberts Corporation.

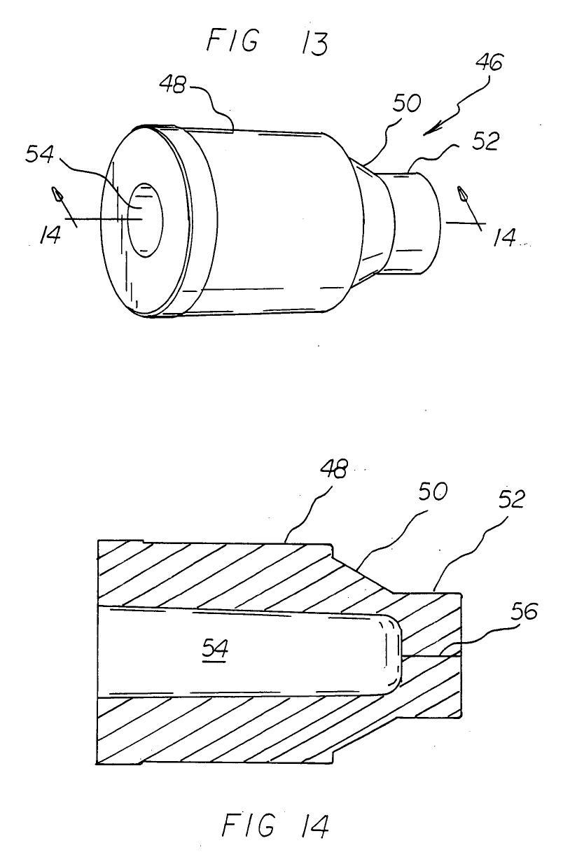

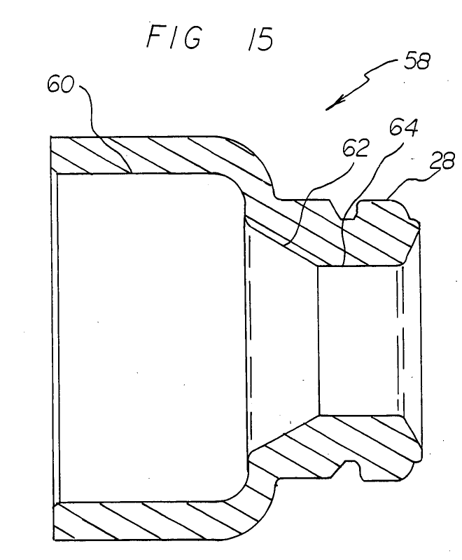

More specifically, referring to FIGS. 13-14, the swabbable valve 26 comprises a valve element 46 having a body portion 48, a frustro-conical portion 50 and a tip portion 52 composed of a resilient material. A central bore 54 extends through the body portion 48 and the frustro-conical portion 50. A diametrical slit 56 is formed in the tip portion 52.

The valve element 46 is inserted into the inlet 20 and held into position by a collar 58 with female luer threads 58 formed on the end thereof for threaded engagement with a male luer fitting (see FIG. 1). As best shown in FIG. 15, the collar 58 comprises central bore defined by a body portion 60, a frustro-conical portion 62 and a tip portion 64, respectively corresponding to the configuration of the body portion 48, frustro-conical portion 50 and tip portion 52 of the valve element 46.

During assembly, the valve element 46 is inserted into the inlet 20 and the collar 58 is then fitted over the valve element 46 and the lumen of the body portion 60 is affixed to the outside surface of the inlet 20 (e.g., by sonic welding). The relative dimensions are such that once assembled, the tip portion 52 of the valve element 46 is under some compression by the tip portion 64 of the central bore of the female collar 58 to seal the slit 56 closed and to seal the outer surface of the tip portion 52 of the valve element 46 against the lumen of the tip portion 64 of the collar 58, thereby precluding any fluid flow through the swabbable valve.

When the tip of a device such as a syringe is inserted into the collar 58, the resilient valve element 46 is urged inwardly until the tip portion 52 is moved inwardly away from the tip portion 64 of the collar 58, whereupon the syringe tip opens the slit 56 and fluid flow is allowed through the inlet 20.

It should now be appreciated from the foregoing that when the stopcock 10 is connected in-line with an IV line and another fluid is desired to be injected into or otherwise connected in fluid communication with the IV line from another component (e.g., from a syringe with a plain tip or from a luer-fitted syringe or other luer-fitted device such as another IV line), the luer-fitted component is simply inserted into the collar 58 of the swabbable valve 29 to open it allowing fluid flow through inlet 20 into the IV line.

The stopcock 10 functions as follows. As represented by the respective arrows 42, fluid flow is permitted to flow through the valve element 12 when two or more of the arrows 42 are aligned with two or more of the three flow channels 30, 32 and 34.

More specifically, in the position of the valve element 12 shown in FIG. 1 with all three arrows 42 aligned with inlets 16, 18 and 20, fluid flow is allowed through inlets 16 and 18 and, if the swabbable valve 26 is actuated open, through inlet 20. Upon turning the valve element 90 degrees clockwise, arrows 42 are aligned only with inlets 18 and 20 and fluid flow is allowed through inlet 18 and, if the swabbable valve 26 is actuated open, through inlet 20. Upon turning the valve element 90 degrees counter-clockwise, arrows 42 are aligned only with inlets 16 and 20 and fluid flow is allowed through inlet 16 and, if the swabbable valve 26 is actuated open, through inlet 20. Upon turning the valve element 180 degrees clockwise (or counter-clockwise), arrows 42 are aligned only with inlets 16 and 18 and fluid flow is allowed only through inlets 16 and 18 even if the swabbable valve 26 is actuated open.

Reference to the male and female fittings are for ease in reference only. It shall be understood that any suitable fitting my be employed as desired.

The present disclosure includes that contained in the appended claims, as well as that of the foregoing description. Although this invention has been described in its preferred form with a certain degree of particularity, it is understood that the present disclosure of the preferred form has been made only by way of example and that numerous changes in the details of construction and the combination and arrangement of parts may be resorted to without departing from the spirit and scope of the invention.

Now that the invention has been described, what is claimed is:

1. a stopcock for connection in-line with a fluid line, comprises in combination:
a main valve body main having at least a plurality of inlets;
a stopcock valve element positioned within said main valve body that controls the flow of fluid through said inlets; and
a swabbable valve fitted to one of said inlets.

2. The stopcock as set forth in claim 1, wherein said inlets comprise two said inlets positioned in-line.

3. The stopcock as set forth in claim 1, wherein at least one of said inlets comprises a valve.

4. The stopcock as set forth in claim 3, wherein said valve comprises a swabbable valve.

5. The stopcock as set forth in claim 4, wherein said inlets comprise two said inlets positioned in-line and wherein said inlets comprising said swabbable valve is positioned between said in-line inlets.

6. The stopcock as set forth in claim 5, wherein said swabbable valve inlet is positioned transversely between said in-line inlets.

9. The stopcock as set forth in claim 1, wherein at least one of said inlets comprises a female luer fitting.

10. The stopcock as set forth in claim 1, wherein at least one of said inlets comprises a male luer fitting.

11. The stopcock as set forth in claim 1, wherein said stopcock valve element comprises an elongated circular cylindrical configuration having flow channels corresponding to respective said inlets.

12. The stopcock as set forth in claim 11, wherein said flow channels are formed in a lower portion said elongated circular configuration in a position such that when said stopcock valve element is positioned fully into said main body element, the plane of said flow channels are alignment with the plane of said inlets.

13. The stopcock as set forth in claim 12, wherein said valve element comprises a plurality of handles extending from an upper portion of said elongated circular cylindrical configuration in radial alignment with respective said flow channels.
